(19) United States
(12) Patent Application Publication
(10) Pub. No.: US 2025/0025103 A1
(43) Pub. Date: Jan. 23, 2025

(54) ORAL SENSOR SYSTEM

(71) Applicant: Ivoclar Vivadent AG, Schaan (LI)

(72) Inventors: Marvin KUHN, Feldkirch (AT); Alexander Albert SKAWRAN, Sargans (CH); Christian NIEDRIG, Azmoos (CH); Alexander GROSSE-HONEBRINK, Oberriet (CH); Florian GEISTER, Basel (CH); Nicolas GLASER, Riehen (CH)

(21) Appl. No.: 18/777,924

(22) Filed: Jul. 19, 2024

(30) Foreign Application Priority Data
Jul. 20, 2023 (EP) 23186732.6

(51) Int. Cl. A61B 5/00

(52) U.S. Cl. CPC A61B 5/682; A61B 2560/0443

(57) ABSTRACT

An oral sensor system (100) having a housing (101) with an electrical circuit (103) for evaluating measurement signals. It further includes a replaceable contact device (105) for the electrical circuit (103), which is coupled to the housing (101) in a liquid-tight manner.

### CROSS-REFERENCE TO RELATED APPLICATIONS

This application claims priority to European Patent Application No. 23186732.6 filed on Jul. 20, 2023, the disclosure of which is incorporated herein by reference in its entirety.

### TECHNICAL FIELD

The present invention relates to an oral sensor system.

### BACKGROUND

Current oral sensor systems are difficult to handle as they are too large or require additional tools. Molded-in electrics are only suitable for single use.

US 2019083215 and 2010130837 are directed to monitoring devices and are hereby incorporated by reference in their entirety.

### SUMMARY

It is the technical task of the present invention to provide an efficient and modular oral sensor system with easy handling.

This task is solved by subject matter according to the independent claims. Technically advantageous embodiments are the subject matter of the dependent claims, the description and the drawings.

According to a first aspect, the technical task is solved by an oral sensor system comprising a housing with an electrical circuit for evaluating measurement signals; and a replaceable contact device for the electrical circuit, which is coupled to the housing in a liquid-tight manner. The oral sensor system is used to determine measurement data within a patient's oral cavity. The contact device comprises, for example, a biochemical sensor. The contact device comprises, for example, a printed circuit board or a screen-printed foil.

In a technically advantageous embodiment of the oral sensor system, the replaceable contact device comprises an adhesive and/or swelling layer for liquid-tight coupling with the housing. This provides the technical advantage, for example, that a good seal is created between the housing and the contact device.

In a further technically advantageous embodiment of the oral sensor system, the adhesive and/or swelling layer comprises hydrocolloids. This provides the technical advantage, for example, that the seal is improved when in contact with moisture.

In a further technically advantageous embodiment of the oral sensor system, the adhesive and/or swelling layer is arranged around the contact surfaces on the contact device. This provides the technical advantage, for example, that the contact surfaces are protected from moisture.

In a further technically advantageous embodiment of the oral sensor system, the contact device has a disk shape, a plate shape or a cube shape. The contact device can also have another shape to which conducting tracks can be attached, such as rectangular or square. This provides the technical advantage, for example, that space-saving contact devices can be used for an oral application.

In a further technically advantageous embodiment of the oral sensor system, the contact device has contact surfaces on one and/or the other side. This provides the technical advantage, for example, that the contact device can be used on one or both sides or for through-connection.

In a further technically advantageous embodiment of the oral sensor system, the housing comprises a recess for inserting the contact device. This provides the technical advantage, for example, that the contact device can be accommodated by the housing.

In a further technically advantageous embodiment of the oral sensor system, the housing or the contact device comprises a sealing ring for sealing the contact device or the housing in the recess. This provides the technical advantage, for example, of preventing moisture from entering the recess.

In a further technically advantageous embodiment of the oral sensor system, an edge of the contact device is made of plastic. The plastic may comprise an elastomer or rubber. This provides the technical advantage, for example, that the contact device can be manufactured in a simple manner.

In a further technically advantageous embodiment of the oral sensor system, the housing and/or the contact device comprises a screw thread. This provides the technical advantage, for example, that the contact device can be firmly attached.

In a further technically advantageous embodiment of the oral sensor system, the contact device comprises a sensor base protruding from a sealing material. This provides the technical advantage, for example, that a reliable contact can be established.

In a further technically advantageous embodiment of the oral sensor system, the sealing material comprises a recess for inserting a housing portion. This provides the technical advantage, for example, that the contact device can be attached quickly and easily.

In a further technically advantageous embodiment of the oral sensor system, the housing portion comprises a protruding collar.

This provides the technical advantage, for example, of preventing moisture from entering.

In a further technically advantageous embodiment of the oral sensor system, the recess comprises a circumferential depression for receiving the collar. This provides the technical advantage, for example, of preventing moisture from entering even better.

In a further technically advantageous embodiment of the oral sensor system, the sealing material is made of rubber or silicone. This provides the technical advantage, for example, of achieving a good sealing effect.

### BRIEF DESCRIPTION OF THE DRAWINGS

Exemplary embodiments of the invention are shown in the drawings and are described in more detail below, in which:

FIG. 1 shows a perspective view and a cross-sectional view of a contact device;

FIG. 2A shows schematic views of another embodiment of a contact device;

FIG. 2B shows schematic views of another embodiment of a contact device;

FIG. 3 shows schematic views of further embodiments of the contact device;

FIG. 4 shows a schematic view of another oral sensor system;

FIG. 5 shows a schematic view of another oral sensor system;

FIG. 6 shows a schematic view of another oral sensor system; and

FIG. 7 shows a schematic view of another oral sensor system.

### DETAILED DESCRIPTION

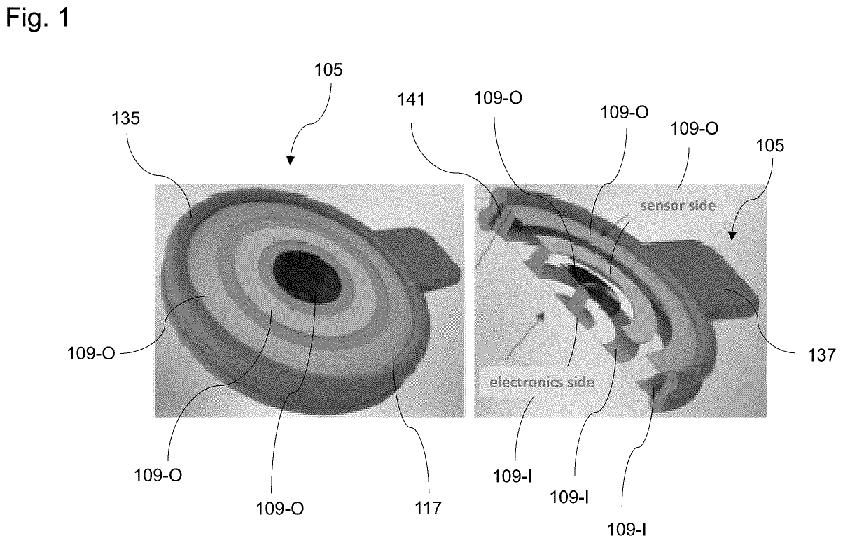

FIG. 1 shows a perspective view and a cross-sectional view of a replaceable and disk-shaped contact device 105 for an oral sensor system. The contact device 105 is, for example, a replaceable contact element. The contact device 105 may comprise, for example, a biochemical sensor for oral applications. The oral sensor system is used to obtain measurement data from the mouth of a patient, such as lactate or pH values.

The contact device 105 serves to establish an electrical contact between a housing, in which an electrical circuit for evaluating measurement signals is arranged, and a contact surface 109-O of the contact device 105, which is in contact with the oral environment. The electrical circuit generates suitable currents and voltages, which are emitted to the environment via the contact device 105. The replaceable contact device 105 realizes a reversible sensor sealing system for water-tight attachment of a measuring electrode to the electrical circuit.

For this purpose, the contact device 105 has ring-shaped contact surfaces 109-O on an outside, which are mutually separated from one another. The contact surfaces 109-O on the outside form sensor electrodes and are connected to the contact surfaces 109-I on the inside via through-contacts 141 through the disk- or plate-shaped contact device 105. The contact surfaces 109-I on the inside are in electrical contact with the electrical circuit. A carrier or edge 117 of the contact device 105 can be made of plastic or ceramic.

The insertable contact device 105 achieves a water-tight seal with an electrical circuit for evaluating measurement signals. For this purpose, the insertable contact device 105 is manufactured with a laterally circumferential elastomer sealing 135. The contact device 105 is molded with an elastomer in such a way that it can be inserted into the counterpart of the housing in a water-tight manner. The contact device 105 additionally comprises a lug 137 on one side, which makes it easy to remove and replace.

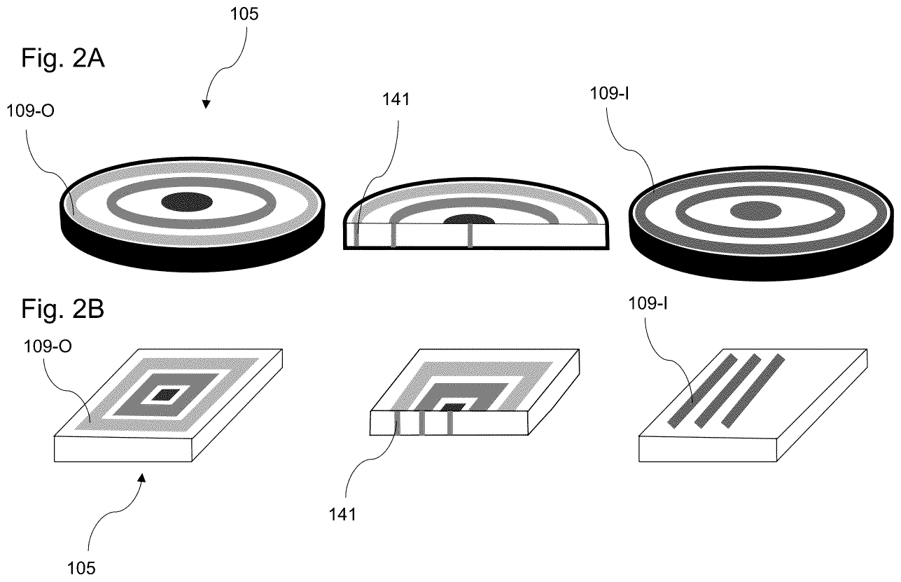

FIG. 2A shows schematic views of the disk-shaped contact device 105. The outside of the contact device 105 has ring-shaped contact surfaces 109-O, which are mutually separated from one another. The ring-shaped contact surfaces 109-O are connected to ring-shaped contact surfaces 109-I on the inside of the contact device 105 via through-contacts 141.

FIG. 2B shows schematic views of another embodiment of a rectangular contact device 105. The outside of the contact device 105 has rectangular contact surfaces 109-O, which are mutually separated from one another. The rectangular contact surfaces 109-O are connected to strip-shaped contact surfaces 109-I on the inside of the contact device 105 via through-contacts 141.

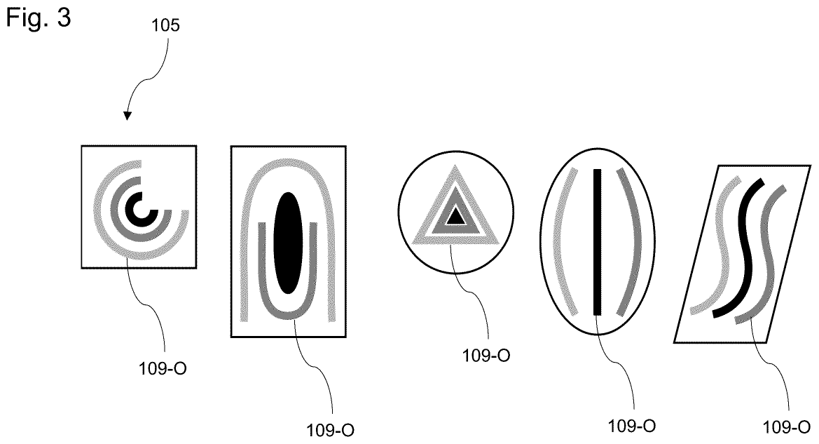

FIG. 3 shows schematic views of further embodiments of the contact device 105. The contact device 105 can have different shapes, such as square, rectangular, circular, oval or trapezoidal. On the outside of the contact device 105 there are differently shaped contact surfaces 109-O, such as circular segment-shaped, U-shaped, triangular, curved or S-shaped. The shapes of the contact device 105 and the shapes of the contact surfaces 109-O can be combined in any way.

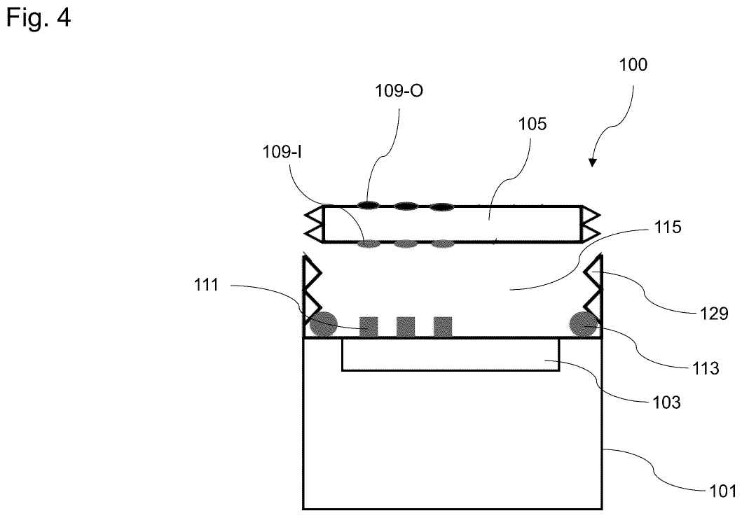

FIG. 4 shows a schematic view of the replaceable contact device 105 in the housing 101 of the oral sensor system 100. The housing 101 comprises the electrical circuit 103 for evaluating measurement signals. The contact device 105 is contacted by a contact pressure connection on the inner side, so that an electrical connection is established between the corresponding contact surfaces 111 of the electrical circuit 103 of the housing 101 and the contact surfaces 109-I of the contact device 105. The contact surfaces 111 of the housing 101 can be formed by resilient contact pins. The contact surfaces 109-O for the electrical measurement of the sensor system 100 are located on the outer side of the contact device 105.

The contact device 105 is attached to the enclosed electrical circuit 103 inside the housing 101 in a water-tight manner by means of a sealing ring 113. The disk-shaped contact device 105 is screwed into a cylindrical recess 115 of the housing 101 by means of a screw thread 129, for example. In this way, the contact device 105 is arranged in the housing 101, attached and contacted with the contact surfaces 111.

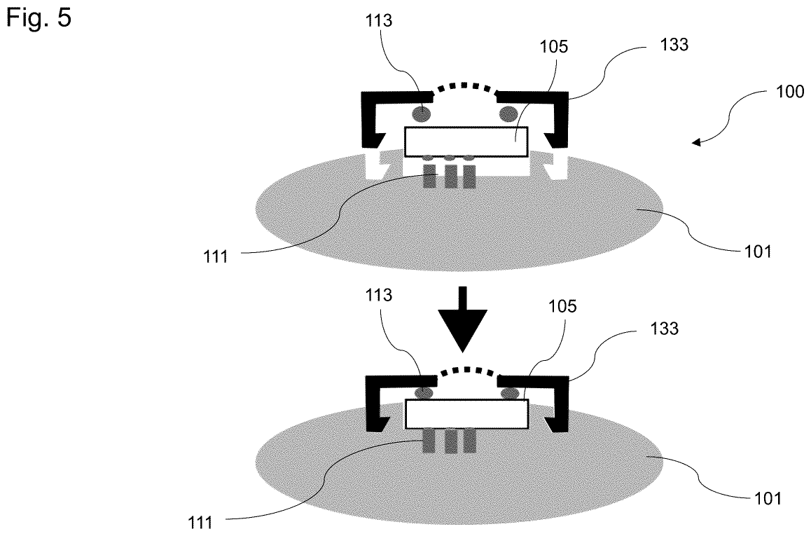

FIG. 5 shows a schematic view of another contact device 105 of the oral sensor system 100. The contact device 105 is held by a reusable click or screw mechanism 133 at the intended connection point of the housing 101. The pot-shaped click or screw mechanism 133 grips around the contact device 105 and engages on the housing 101. To enable the contact surfaces 109-O to come into contact with the environment, the click or screw mechanism 133 comprises an opening. The sealing ring 113 is arranged between the contact device 105 and the click or screw mechanism 133.

In this way, the replaceable contact device 105 can be connected to the sealed electrical circuit 103 in a water-tight manner. This enables a compact design and easy handling of the oral sensor system 100.

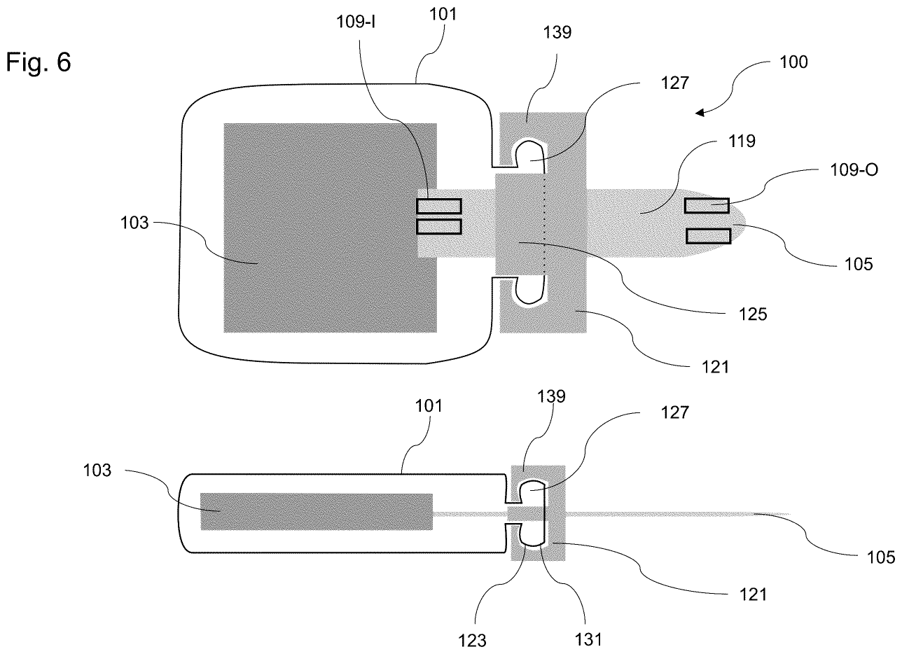

FIG. 6 shows a schematic view of another oral sensor system 100. The contact device 105 is formed by a foil that is molded into or passed through a plug 139. The foil comprises the contact surfaces 109-O. The contact device 105 may comprise a biochemical sensor for oral applications.

The plug 139 is made of rubber or silicone-like material and encloses the foil-like contact device 105 laterally. The contact device 105 is thus surrounded by a seal so that it is sealed water-tight when inserted. The contact surfaces 109-I form a sensor base 119, which protrudes from the sealing material 121 of the plug 139. The sealing material 121 also comprises a recess 123 for inserting a housing portion 125. The recess 123 is formed as a circumferential depression 131 for receiving the collar 127.

The housing 101, which comprises the electrical circuit 103, has a protuberance with a collar 127 as a counterpart. The plug 139 is placed on the collar 127 during insertion. After inserting the foil-like contact device 105 with the plug 139, the opening of the housing 101 is sealed. In this way, the contact device 105 can be plugged into and attached to the sealed electrical circuit 103 in a water-tight manner. This reduces the effort involved in the production of the oral sensor system 100 and enables a compact design and easy handling.

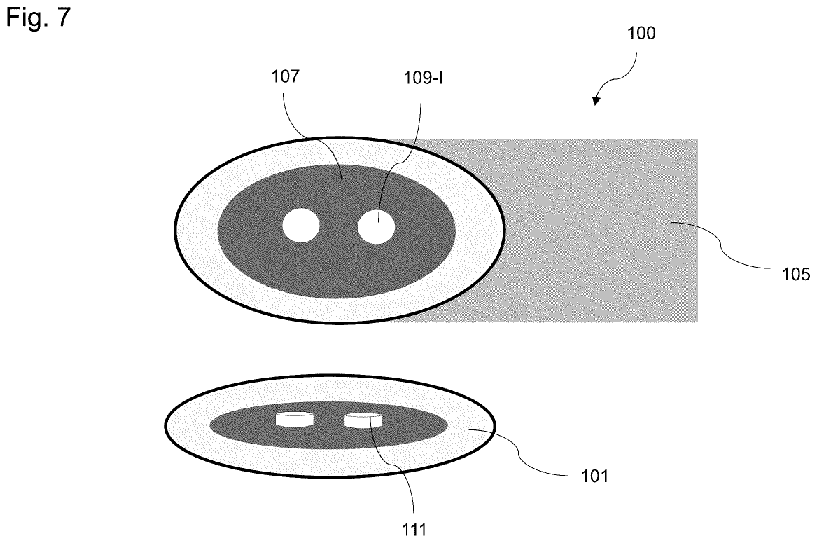

FIG. 7 shows a schematic view of another contact device 105. The foil-like contact device 105 has two inner contact surfaces 109-I arranged next to each other. The inner contact surfaces 109-I are surrounded by a layer 107. The layer 107 consists of a material which is swellable and adhesive in conjunction with moisture and which can seal unevenness or damage. The layer 107 runs around the imprinted contact surfaces 109-I of the contact device 105.

When hydrocolloids are used in the layer 107, the coating swells on contact with water or saliva, for example, so that the sealing effect is increased. Hydrocolloids dissolve in water as colloids and have a high capacity for gel formation. Hydrocolloids are formed from polysaccharides and proteins, for example. Thus, the adhesive and swelling coating creates protection against moisture penetration.

The layer 107 is thus connected to the housing 101 or glued to the housing 101 in a water-tight manner. An unused layer can be used each time the contact device 105 is replaced. This ensures that the transition to the housing 101 is tight. Used layers 107 can simply be wiped off if the connection surface is flat. Minor residues or dirt can be bridged with a new, swelling layer 107. In this way, a water-tight electrical contact can be achieved between the contact surfaces 109-I of the contact device 105 and the contact surfaces 111 of the electrical circuit 103. The adhesive connection is thus made from a coating that can bridge small gaps. The contact device 105 is attached to the electrical circuit 103 by means of an adhesive connection on the housing 101.

The contact device 105 is glued to the housing 101 with a flat surface and protruding contacts of the electrical circuit 103. Due to its small size, the contact device 105 is easy to replace without the need for miniaturized mechanical components. This achieves a water-tight connection between the contact device 105 for oral applications and the electrical circuit 103. This reduces the effort involved in the production of the oral sensor system 100 and enables a compact design and easy handling.

All the features explained and shown in connection with individual embodiments of the invention can be provided in different combinations in the subject matter according to the invention in order to simultaneously realize their advantageous effects.

All method steps can be implemented by devices that are suitable for executing the respective method step. All functions performed by the features of the subject matter can be a method step of a method.

The scope of protection of the present invention is given by the claims and is not limited by the features explained in the description or shown in the figures.

### REFERENCE LIST

100 Oral sensor system
101 Housing
103 Circuit
105 Contact device
107 Swelling layer
109 Contact surface
111 Contact surface
113 Sealing ring
115 Recess
117 Edge
119 Sensor base
121 Sealing material
123 Recess
125 Housing portion
127 Collar
129 Screw thread
131 Depression
133 Click or screw mechanism
135 Elastomer sealing
137 Lug
139 Plug
141 Through-contact

1. An oral sensor system, comprising:
a housing with an electrical circuit for evaluating measurement signals; and
a replaceable contact device for the electrical circuit, which is coupled to the housing in a liquid-tight manner.

2. The oral sensor system according to claim 1,
wherein the replaceable contact device comprises an adhesive and/or swelling layer for liquid-tight coupling with the housing.

3. The oral sensor system according to claim 2,
wherein the adhesive and/or swelling layer comprises hydrocolloids.

4. The oral sensor system according to claim 2,
wherein the adhesive and/or swelling layer is arranged around the contact surfaces on the contact device.

5. The oral sensor system according to claim 1,
wherein the contact device has a disk shape, a plate shape or a cube shape.

6. The oral sensor system according to claim 5,
wherein the contact device has contact surfaces on one and/or the other side.

7. The oral sensor system according to claim 5,
wherein the housing comprises a recess for inserting the contact device.

8. The oral sensor system according to claim 7,
wherein the housing or the contact device comprises a sealing ring for sealing the contact device or the housing in the recess.

9. The oral sensor system according to claim 5,
wherein an edge of the contact device is made of plastic.

10. The oral sensor system according to claim 5,
wherein the housing and/or the contact device comprises a screw thread.

11. The oral sensor system according to claim 1,
wherein the contact device comprises a sensor base protruding from a sealing material.

12. The oral sensor system according to claim 11,
wherein the sealing material comprises a recess for inserting a housing portion.

13. The oral sensor system according to claim 12,
wherein the housing portion comprises a protruding collar.

14. The oral sensor system according to claim 11,
wherein the recess comprises a circumferential depression for receiving the collar.

15. The oral sensor system according to claim 11,
wherein the sealing material is made of rubber or silicone.
